(19) United States
(12) Patent Application Publication
(10) Pub. No.: US 2004/0171864 A1
(43) Pub. Date: Sep. 2, 2004

(54) Fluorinated aromatics

(76) Inventors: Karen Peilstocker, Koln (DE); Albrecht Marhold, Leverkusen (DE); Axel Pleschke, Koln (DE); Guido Giffels, Bonn (DE)

Correspondence Address: LANXESS CORPORATION, PATENT DEPARTMENT/ BLDG 14, 100 BAYER ROAD, PITTSBURGH, PA, 15205-9741, US

(21) Appl. No.: 10/719,626

(22) Filed: Nov. 21, 2003

(30) Foreign Application Priority Data
Dec. 9, 2002 (DE) 10257356.5

(51) Int. Cl. C07C 253/02

(52) U.S. Cl. 558/388

(57) ABSTRACT

The present invention relates to fluorinated aromatics, to a process for preparing them and also to the use of the fluorinated aromatics for preparing active ingredients, especially in medicaments and agrochemicals.

### BACKGROUND OF THE INVENTION

1. Field of the Invention

The present invention relates to fluorinated aromatics, to a process for preparing them and also to the use of the fluorinated aromatics for preparing active ingredients, especially in medicaments and agrochemicals.

2. Brief Description of the Prior Art

Fluorinated aromatics, especially fluorinated phenols and fluorinated phenol ethers, are valuable starting materials for the preparation of active ingredients in medicaments and agrochemicals. Their fluorine or fluorinated substituents increase the lipophilicity and therefore the ability of the entire active ingredient molecule to pass through membranes. For example, substituted, fluorinated methoxyarylacetonitriles are particularly suitable as the starting material for preparing medicaments which are used for treating cardiovascular diseases (see also WO-A 01/19780).

The methods of preparing the fluorinated aromatics and the attendant disadvantages are described as follows. Illustratively 5-fluoro-2-hydroxyphenylacetonitrile can be prepared, for example, starting from 5-fluoro-2-hydroxybenzaldehyde by a four-stage synthesis sequence (WO-A 01/19780, p. 81 ff). A disadvantage of this method is that the synthesis sequence is very long and starts from a reactant which can itself be prepared in a complicated manner or in very low yields by formylating p-fluorophenol (Suzuki et al., Chem. Pharm. Bull. 1963, 31(5), 1751-1753).

There is therefore a need to provide a process which enables the preparation of fluorinated phenylacetonitriles in good yields and in a simple manner.

### SUMMARY OF THE INVENTION

A process has now been found for preparing compounds of the formula (I)

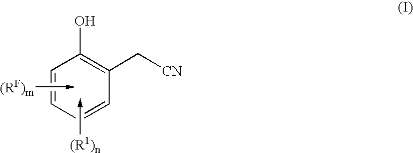

where

R¹ is in each case independently C₁-C₁₂-alkyl, free or protected formyl, chlorine or bromine or a radical of the formulae (IIa) or (IIb)

A-B-D-E (IIa)

A-E (IIb)

where, each independently,

A is absent or is a C₁-C₈-alkylene radical and

B is absent or is oxygen, sulphur or NR²

where R² is hydrogen or C₁-C₈-alkyl and

D is a carbonyl group and

E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and

n is an integer of 0 to 4-m and

R^{F} is fluorine, C₁-C₁₂-fluoroalkyl, —O(C₁-C₁₂-fluoroalkyl) or —S(C₁-C₁₂-fluoroalkyl) and

m is an integer of 1 to 3,

which is characterized in that

a) compounds of the formula (II)

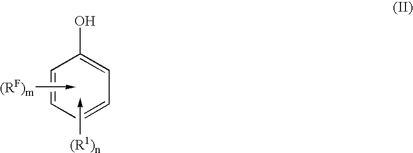

where R¹ and R^{F}, and also n and m, are as defined above are converted

in the presence of formaldehyde and

in the presence of secondary amines of the formula (III)

HNR³R⁴ (III)

where R³ and R⁴ are each independently C₁-C₈-alkyl, or NR³R⁴ as a whole is a cyclic amino radical having a total of 4 to 12 carbon atoms

to compounds of the formula (IV)

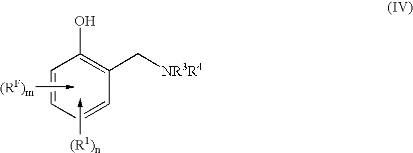

where R¹, R³, R⁴ and R^{F}, and also m and n, are as defined above and

b) the compounds of the formula (IV) are converted by reacting them with compounds of the formula (V)

R⁵CO—O—OCR⁵ (V)

where the R⁵ radicals are each independently hydrogen, C₁-C₁₂-alkyl, C₂-C₁₂-alkenyl, C₅-C₁₄-aryl or C₆-C₁₅-arylalkyl

to compounds of the formula (VI)

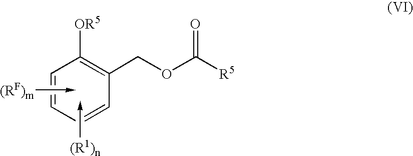

and

c) the compounds of the formula (VI) are converted to compounds of the formula (I) by reacting them with cyanide.

Within the scope of the invention, all of the radical definitions, parameters and illustrations hereinabove and cited hereinbelow, specified in general or within preferred ranges i.e. the particular areas and areas of preference, may be combined as desired.

### DETAILED DESCRIPTION OF THE INVENTION

Alkyl, alkylene, alkoxy and alkenyl are in each case independently a straight-chain, cyclic, branched or unbranched alkyl, alkylene, alkoxy and alkenyl radical respectively. The same applies to the nonaromatic moiety of an arylalkyl radical.

C₁-C₄-Alkyl is, for example, methyl, ethyl, n-propyl, isopropyl, n-butyl, sec-butyl and tert-butyl, C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl, and C₁-C₁₂-alkyl is still further additionally, for example, adamantyl, n-nonyl, n-decyl and n-dodecyl.

C₁-C₈-Alkoxy is, for example, methoxy, ethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cyclohexoxy, cyclopentoxy, n-hexoxy and n-octoxy.

Fluoroalkyl is in each case independently a straight-chain, cyclic, branched or unbranched alkyl radical which is substituted by at least one fluorine atom and optionally further by chlorine atoms and/or bromine atoms.

C₁-C₁₂-Polyfluoroalkyl is, for example, trifluoromethyl, chlorofluoromethyl, difluoromethyl, difluorochloromethyl, 1,1,2,2-tetrafluoro-1-ethyl, 2-chloro-2,1,1-trifluoro-1-ethyl, 2,2,2-trifluoroethyl, pentafluoroethyl, 1,1-dichloro-2,2,2-trifluoroethyl, heptafluoroisopropyl, n-nonafluorobutyl, perfluorocyclopentyl, perfluorocyclohexyl and perfluorododecyl.

Aryl is in each case independently a heteroaromatic radical having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen, but is preferably a carbocyclic aromatic radical having 6 to 14 framework carbon atoms.

Examples of carbocyclic aromatic radicals having 6 to 14 framework carbon atoms are phenyl, naphthyl, phenanthrenyl, anthracenyl or fluorenyl, and heteroaromatic radicals having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

Moreover, the carbocyclic aromatic radical or heteroaromatic radical may be substituted by up to five identical or different substituents per cycle which are selected from the group of chlorine, fluorine, C₁-C₁₂-alkyl, C₁-C₁₂-perfluoroalkyl, COO(C₁-C₈-alkyl), CON(C₁-C₈-alkyl)₂, COO(C₁-C₈-arylalkyl), COO(C₄-C₁₄-aryl), CO(C₁-C₈-alkyl), C₅-C₁₅-arylalkyl or tri(C₁-C₆-alkyl)siloxyl.

Arylalkyl is in each case independently a straight-chain, cyclic, branched or unbranched alkyl radical which may be singly, multiply or fully substituted by aryl radicals as defined above.

C₆-C₁₅-Arylalkyl is, for example and with preference, benzyl.

The preferred substitution patterns for compounds of the formulae (I) to (VI) are defined hereinbelow:

R¹ is preferably in each case independently C₁-C₄-alkyl, free or protected formyl or chlorine, more preferably methyl.

n is preferably 0 or 1, more preferably 0.

R^{F} is preferably fluorine, C₁-C₄-fluoroalkyl, —O(C₁-C₄-fluoroalkyl) or —S(C₁-C₄-fluoroalkyl), more preferably trifluoromethyl, trifluoromethylthio, trifluoromethoxy, chlorofluoromethyl, chlorofluoromethylthio, chlorofluoromethoxy, difluoromethoxy, difluoromethyl, difluoromethylthio, difluoromethoxy, difluorochloromethyl, difluorochloromethylthio, difluorochloromethoxy, 1,1,2,2-tetrafluoro-1-ethyl, 1,1,2,2-tetrafluoro-1-ethylthio, 1,1,2,2-tetrafluoro-1-ethoxy, 2-chloro-2,1,1-trifluoro-1-ethyl, 2-chloro-2,1,1-trifluoro-1-ethylthio, 2-chloro-2,1,1-trifluoro-1-ethoxy, 2,2,2-trifluoroethyl, 2,2,2-trifluoroethylthio, 2,2,2-trifluoroethoxy, pentafluoroethyl, pentafluoroethylthio, pentafluoroethoxy, 1,1-dichloro-2,2,2-trifluoroethyl, 1,1-dichloro-2,2,2-trifluoroethylthio, 1,1-dichloro-2,2,2-trifluoroethoxy, heptafluoroisopropyl, n-nonafluorobutyl, perfluorocyclopentyl, perfluorocyclohexyl and perfluorododecyl.

m, in the case that all R^{F} are fluorine, is an integer of 1 to 3, otherwise one or two, preferably one.

R³ and R⁴ are preferably each an identical C₁-C₈-alkyl radical, more preferably identically methyl or ethyl.

R⁵ is preferably in each case identically hydrogen, C₁-C₁₂-alkyl, C₂-C₁₂-alkenyl, C₅-C₁₄-aryl or C₆-C₁₅-arylalkyl, more preferably methyl.

In step a), the compounds of the formula (II) are converted in the presence of formaldehyde and in the presence of secondary amines of the formula (III) to compounds of the formula (IV).

The molar ratio of formaldehyde to compounds of the formula (II) may be, for example, 0.8 to 10, preferably 1.0 to 10 and more preferably 1.2 to 3.6. The molar ratio of secondary amines of the formula (III) to compounds of the formula (II) may be, for example, 0.8 to 10, preferably 1.0 to 10 and more preferably 1.05 to 3.15.

Formaldehyde can be used, for example, as paraformaldehyde or in the form of an aqueous solution, preferably in the form of a 32 to 40% by weight solution.

The secondary amines of the formula (III) can be used, for example, without solvent or, if possible, in the form of aqueous solutions. Particular preference is given to using dimethylamine in the form of an aqueous solution.

The reaction temperature may be, for example, −20° C. to 120° C., preferably −10 to 40° C. and more preferably −5 to 10° C.

The reaction pressure may be, for example, 0.5 to 100 bar, although preference is given to ambient pressure.

The reaction time may be 10 min to 72 hours, preferably 3 hours to 24 hours.

The procedure for converting the compounds of the formula (II) is, for example, that the compounds of the formula (II) and the secondary amines of the formula (III) are initially charged and the formaldehyde is subsequently added.

The compounds of the formula (IV) can be worked up in a manner known per se by extraction and subsequent distillation or, in the case of compounds of the formula (IV) which are solid at 30° C., by recrystallization.

As important intermediates, the compounds of the formula (IV) are likewise encompassed by the invention, with the exception of 2-hydroxy-5-fluoro-N,N-dimethylbenzylamine

Particularly preferred individual compounds of the formula (IV) include: 4,5-difluoro-2-hydroxy-N,N-dimethylbenzylamine, 2-hydroxy-5-(trifluoromethoxy)-N,N-dimethylbenzylamine, 6-hydroxy-2,3,4-trifluoro-N,N-dimethylbenzylamine and 2-hydroxy-4-(trifluoromethyl)-N,N-dimethylbenzylamine.

The starting compounds of the formula (II) required for step a) are known in the art or can be synthesized in a similar manner as described in the art.

It is preferred to prepare compounds of the formula (Ia)

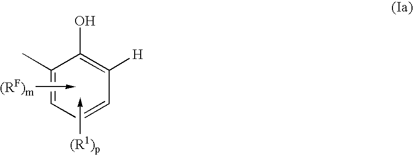

where R¹, R^{F} and m each have the definition and areas of preference specified above and

p is an integer between 0 and 3-m,

by reducing compounds of the formula (IVa)

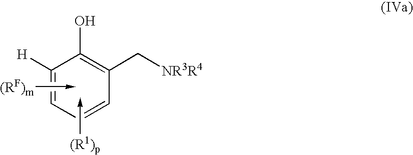

where R¹, R³, R⁴ and R^{F}, and also p and m, each have the definition and areas of preference specified above.

The reduction may advantageously be carried out in the presence of hydrogen and hydrogenation catalyst.

Preferred hydrogenation catalysts are, for example, metals or metal compounds such as salts or complexes of nickel, palladium, platinum, cobalt, rhodium, iridium and ruthenium, although preference is given to metals such as nickel or palladium. Particular preference is given to using metals in finely divided form, for example as Raney metal or applied to a support material.

Particular preference is given to carrying out the reduction with hydrogen and Raney nickel and/or palladium on carbon.

The reduction may, for example, be carried out at a reaction temperature of 20° C. to 200° C., preferably 50 to 180° C. and more preferably 80 to 150° C.

The partial hydrogen pressure in the reduction may be, for example, 0.1 to 180 bar, preferably 10 to 150 bar and more preferably 40 to 120 bar.

Optionally and with preference, the reduction may be carried out in the presence of solvent, as long as it is substantially inert under the selected reaction conditions.

Suitable solvents are, for example, aliphatic, alicyclic or aromatic, optionally halogenated hydrocarbons, for example benzine, benzene, toluene, xylene, chlorobenzene, dichlorobenzene, petroleum ether, hexane, cyclohexane, dichloromethane, chloroform or carbon tetrachloride; ethers such as diethyl ether, diisopropyl ether, dioxane, tetrahydrofuran or ethylene glycol dimethyl or diethyl ether; alcohols, for example methanol, ethanol and isopropanol, carboxylic acids, for example acetic acid, or mixtures of solvents.

The reaction time in the reduction may be 10 min to 200 hours, preferably 5 to 100 hours.

In a particularly preferred embodiment, the reduction is carried out in the presence of palladium on activated carbon and in the presence of acetic acid at a partial hydrogen pressure of 40 to 120 bar.

As a subclass of the compounds of the formula (IV), the compounds of the formula (IVa) are likewise encompassed by the invention.

In step b), the compounds of the formula (IV) are converted to compounds of the formula (VI) with compounds of the formula (V).

The molar ratio of compounds of the formula (V) to compounds of the formula (IV) may be, for example, 1.5 to 10, preferably 2 to 5 and more preferably 2 to 4.

The reaction temperature may be, for example, 0° C. to 200° C., preferably 50 to 150° C. and more preferably 70 to 120° C.

the reaction pressure may be, for example, 0.5 to 100 bar, although preference is given to ambient pressure.

The reaction time may be 10 min to 72 hours, preferably 3 hours to 8 hours.

Optionally, the reaction in step b) may be carried out in the presence of an aprotic organic solvent.

For the purposes of the invention, aprotic means that the organic solvent has no protons which, based on an aqueous comparative scale at 25° C., have a pKa value of below 23.

Suitable organic solvents are, for example, aliphatic or aromatic, optionally halogenated hydrocarbons, for example benzine fractions, benzene, toluene, xylene, chlorobenzene, dichlorobenzene, trichlorobenzene, chlorobenzotrifluorides, petroleum ether, hexane, cyclohexane, dichloromethane, chloroform or carbon tetrachloride, ethers, for example diethyl ether, diisopropyl ether or tert-butyl methyl ether.

The compounds of the formula (VI) can be worked up in a manner known per se by extraction and subsequent distillation or, in the case of compounds of the formula (VI) which are solid at 30° C., by recrystallization.

As important intermediates, the compounds of the formula (VI) are likewise encompassed by the invention, with the exception of 2-acetoxy-5-fluorobenzyl acetate (Synthetic Communications, 2000, 30(3), 397405). The statements made above apply similarly to the areas of preference. Particularly preferred individual compounds of the formula (VI) include: 2-acetoxy-4,5-difluorobenzyl acetate, 2-acetoxy-5-(trifluoromethoxy)benzyl acetate, 6-acetoxy-2,3,4-trifluorobenzyl acetate and 2-acetoxy-4-trifluoromethylbenzyl acetate.

In step c), compounds of the formula (VI) are reacted with cyanide to give compounds of the formula (I).

Useful cyanide sources are, for example, alkali metal cyanides, especially sodium cyanide and potassium cyanide.

The molar ratio of cyanide to compounds of the formula (VI) may be, for example, 0.8:1 to 10:1, preferably 2:1 to 5:1 and more preferably 2.3:1 to 2.7:1.

The reaction temperature may be, for example, −20° C. to 100° C., preferably 20 to 65° C. and more preferably 20 to 50° C.

Useful solvents in step c) are in particular ethers, for example diethyl ether, diisopropyl ether, dioxane, tetrahydrofuran or ethylene glycol dimethyl or diethyl ether; ketones, for example acetone, butanone or methyl isobutyl ketone; nitriles, for example acetonitrile, propionitrile or benzonitrile; amides, for example N,N-dimethylformamide, N,N-dimethylacetamide, N-methylformanilide, N-methylpyrrolidone or hexamethylphosphoramide; esters, for example methyl formate, methyl acetate or ethyl acetate, or alcohols, for example methanol, ethanol, n-propanol, isopropanol and tert-butanol, and also any desired mixtures of such solvents.

The compounds of the formula (I) are likewise encompassed by the invention. Individual compounds of the formula (I) include: 2-hydroxy-5-fluorophenylacetonitrile, 2-hydroxy-4,5-difluorophenyl-acetonitrile, 2-hydroxy-5-trifluoromethoxyphenylacetonitrile, 6-hydroxy-2,3,4-trifluorophenylacetonitrile and 2-hydroxy-4-trifluoromethylphenyl-acetonitrile.

Optionally, compounds of the formula (I) can be reacted in a step d) with compounds of the formulae (VIIa) or (VIIb)

R⁵CO—X (VIIa)

R⁶—Y (VIIb)

where, in formula (VIIa),

R⁵ is hydrogen, C₁-C₁₂-alkyl, C₂-C₁₂-alkenyl, C₅-C₁₄-aryl, C₆-C₁₅-arylalkyl, O(C₁-C₁₂-alkyl), O(C₅-C₁₄-aryl), O(C₆-C₁₅-arylalkyl), O(C₂-C₁₂-alkenyl), NH(C₁-C₁₂-alkyl), NH(C₅-C₁₄-aryl), NH(C₆-C₁₅-arylalkyl), N(C₁-C₁₂-alkyl)₂, N(C₅-C₁₄-aryl)₂ or N(C₆-C₁₅-arylalkyl)₂, preferably C₁-C₁₂-alkyl and more preferably methyl, and

X is OCOR⁵, fluorine, chlorine, bromine or iodine, and

where, in formula (VIIb),

R⁶ is C₁-C₁₂-alkyl, C₅-C₁₄-aryl or C₆-C₁₅-arylalkyl, preferably C₁-C₁₂-alkyl and more preferably methyl, and

Y is O₃SR⁷, chlorine, bromine or iodine where R⁷ is C₁-C₁₂-alkyl, C₅-C₁₄-aryl or C₁-C₁₂-fluoroalkyl,

to give compounds of the formula (VIII)

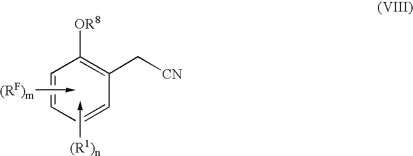

where

R⁸ is R⁵CO or R⁶ as defined above.

This acylation or alkylations can be carried out in a manner known per se, for example in the presence of a base.

Compounds of the formula (VIIIa)

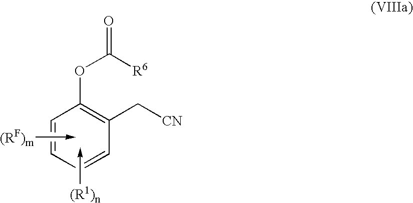

where R¹, R⁶ and R^{F}, and also m and n, each have the definitions and areas of preference specified above, and which are a subclass of the compounds of the formula (VIII), are likewise encompassed by the invention.

The invention also encompasses processes which comprise at least one of the steps a) for preparing compounds of the formula (IV), b) for preparing compounds of the formula (VI), and c) for preparing compounds of the formula (I).

The compounds of the formulae (I) and (VIII) obtainable according to the invention are suitable in particular in a process for preparing active ingredients, for example active ingredients for medicaments. Preferred active ingredients for medicaments are those which are used for treating cardiovascular diseases such as described in WO-A 01/19780.

A substantial advantage of the invention is that the compounds of the formula (I) can be prepared in a simple manner from readily available reactants. Moreover, the compounds of the formulae (I), (IV), (VI) and (VIII) according to the invention constitute valuable starting materials for the preparation of active ingredients, especially for medicaments.

The invention is further described by the following non-limiting examples.

### EXAMPLES

### Example 1

### Preparation of 4,5-difluoro-2-hydroxy-N,N-dimethylbenzylamine

400 g of 3,4-difluorophenol are initially charged in 408 ml of 40% aqueous dimethylamine solution and cooled to 0° C. At 0-5° C., 276 ml of 37% aqueous formaldehyde solution are added dropwise within 60 min. The mixture is kept at 5-10° C. for 2 hours and subsequently stirred at room temperature for 20 hours. The mixture is admixed with 600 ml of water. The organic phase is removed, the aqueous phase is extracted twice with dichloromethane, the combined organic phases are dried and the solvent is distilled off under reduced pressure. The crude product is subsequently fractionally distilled under reduced pressure. 395 g (65% of theory) of a colourless liquid having a boiling point of 93° C. at 16 mbar are obtained.

The ¹H NMR spectrum contained the following characteristic absorptions (CDCl₃, δ/ppm): 11.32 (s, 1 H, OH); 7.77, 7.60 (2 m, 2 H, H-3, H-6); 3.57 (s, 2 H, CH₂); 2.32 (s, 6 H, N(CH₃)₂). The following spectrum was obtained by GC-MS (El, 70 eV, I/%): 187 (100, M⁺); 143 (36, (M-N(CH₃)₂)⁺).

### Example 2

### Preparation of 2-hydroxy-5-(trifluoromethoxy)-N,N-dimethylbenzylamine

130 g of 4-trifluoromethoxyphenol are initially charged in 97 ml of 40% aqueous dimethylamine solution and cooled to 3° C. At 0-5° C., 66 ml of 37% aqueous formaldehyde solution are added dropwise within 45 min.

The mixture is kept at 5-10° C. for a further 2 hours and is subsequently stirred at room temperature for 19 hours. It is cooled again to 0° C., 4.9 ml of 40% aqueous dimethylamine solution are added and 3.3 ml of 37% aqueous formaldehyde solution are subsequently added dropwise. The mixture is stirred at room temperature for a further 3 hours. The mixture is admixed with 100 ml of water. The organic phase is removed, the aqueous phase is extracted twice with dichloromethane, the combined organic phases are dried and the solvent is distilled off under reduced pressure. The crude product is subsequently fractionally distilled under reduced pressure. 118 g (69% of theory) of a light yellow liquid having a boiling point of 110-112° C. at 18 mbar were obtained.

The ¹H NMR spectrum contained the following characteristic absorptions (CDCl₃, δ/ppm): 11.08 (bs, 1 H, OH); 7.03 (dd, 1 H, J_{H4-H3}=8.9 Hz, J_{H4-H6}=2.4 Hz, H-4); 6.84 (d, 1 H, J_{H-6, H-4}=2.3 Hz, H-6); 6.80 (d, 1 H, J_{H3-H4}=8.8 Hz, H-3); 3.62 (s, 2 H, CH₂); 2.32 (s, 3 H, CH₃). The following spectrum was obtained by means of GC-MS (El, 70 eV, I/%): 235 (100, M⁺); 191 (19, (M-N(CH₃)₂)⁺).

### Example 3

### Preparation of 6-hydroxy-2,3,4trifluoro-N,N-dimethylbenzylamine

400 g of 3,4,5-trifluorophenol are initially charged in 359 ml of 40% aqueous dimethylamine solution and cooled to 0C. At 0-5° C., 243 ml of 37% aqueous formaldehyde solution are added dropwise within 90 min. Subsequently, the mixture is stirred at room temperature for 20 hours. The mixture is admixed with 600 ml of water and 500 ml of dichloromethane. The organic phase is removed, the aqueous phase is extracted once with dichloromethane, the combined organic phases are dried and the solvent is distilled off under reduced pressure. The crude product is taken up in 500 ml of water and adjusted to pH 1-2 with cooling in an ice bath using dilute hydrochloric acid. The acidic solution is extracted once with dichloromethane and subsequently adjusted to pH 8-9 with cooling in an ice bath using dilute sodium hydroxide solution. The alkaline reaction solution is extracted three times with dichloromethane, the combined organic phases are washed once with water and dried, and the solvent is removed under reduced pressure. The crude product is subsequently fractionally distilled under reduced pressure. 305 g (55% of theory) of a light yellow liquid having a boiling point of 96° C. at 10 mbar are obtained.

The ¹H NMR spectrum contained the following characteristic absorptions (CDCl₃, δ/ppm): 11.70 (s, 1 H, OH); 6.41 (m, 1 H, H-5); 3.71 (s, 2 H, CH₂); 2.37 (s, 6 H, N(CH₃)₂).

### Example 4

### Preparation of 2-hydroxy-(trifluoromethyl)-N,N-dimethylbenzylamine

195 g of 3-trifluoromethylphenol are initially charged in 160 ml of 40% aqueous dimethylamine solution and cooled to 15° C. At 155° C., 108 ml of 37% aqueous formaldehyde solution are added dropwise within 40 min. Subsequently, the mixture is stirred at room temperature for 20 hours. The mixture is cooled again to 15° C., admixed with 8 ml of 40% aqueous dimethylamine solution and 5.5 ml of 37% aqueous formaldehyde solution are added dropwise. Subsequently, the mixture is stirred at room temperature for a further 4.5 hours. The mixture is cooled again to 15° C. and admixed with 32 ml of 40% aqueous dimethylamine solution, and 21 ml of 37% aqueous formaldehyde solution are added dropwise. Subsequently, stirring is continued at room temperature for 17 hours. The mixture is admixed with 150 ml of water. The organic phase is removed, the aqueous phase is extracted twice with dichloromethane, the combined organic phases are washed once with water and the solvent is distilled off under reduced pressure. The crude product is taken up in 250 ml of water and adjusted to pH 1-2 with cooling in an ice bath using dilute hydrochloric acid. The acidic solution is extracted once with dichloromethane and subsequently adjusted to pH 11 with cooling in an ice bath using dilute sodium hydroxide solution. The alkaline reaction solution is extracted twice with dichloromethane, the combined organic phases are washed once with water and dried, and the solvent is distilled off under reduced pressure. The crude product is subsequently fractionally distilled under reduced pressure. 186 g (71% of theory) of a light yellow liquid having a boiling point of 91° C. at 14 mbar are obtained.

The ¹H NMR spectrum contained the following characteristic absorptions (CDCl₃, δ/ppm): 7.08-6.98 (m, 3 H, H-3, H-5, H-6); 3.67 (s, 2 H, CH₂); 2.32 (s, 6 H, N(CH₃)₂).

### Example 5

### Preparation of 2-acetoxy4,5-difluorobenzyl acetate

200 g of 4,5-difluoro-2-hydroxy-N,N-dimethylbenzylamine are dissolved in 350 ml of toluene and heated to reflux. 252 ml of acetic anhydride are added dropwise and the mixture is stirred at reflux up to complete conversion. The mixture is allowed to cool to 70° C., 100 ml of methanol are added dropwise and the mixture is stirred at 70° C. for 1 hour. After cooling to room temperature, the mixture is washed twice with 5% hydrochloric acid and once with water. The solvent is distilled off under reduced pressure. The crude product is fractionated under reduced pressure. 127 g (48% of theory) of 2-acetoxy-4,5-difluorobenzyl acetate are obtained as a colourless liquid having a boiling point of 119° C. at 2.6 mbar.

The ¹H NMR spectrum contained the following characteristic absorptions (CDCl₃, δ/ppm): 7.28,7.00 (2×m, 2 H, H-3, H-6); 5.00 (s, 2 H, CH2); 2.32, 2.08 (2×s, 6 H, COCH₃).

The following spectrum was obtained by means of GC-MS (El, 70 eV, I/%): 244 (10, M⁺); 202 (100, (M-CH₂CO)⁺); 160 (23, (M-2 CH₂CO)⁺); 142 (92, (202-HOAc)⁺); 114 (45, (142-CO)⁺).

### Example 6

### Preparation of 2-acetoxy-5-(trifluoromethoxy)benzyl acetate

160 g of 2-hydroxy-5-(trifluoromethoxy)-N,N-dimethylbenzylamine are dissolved in 400 ml of toluene and heated to reflux. 160 ml of acetic anhydride are added dropwise and the mixture is stirred at reflux up to complete conversion. The mixture is allowed to cool to 70° C., 100 ml of methanol are added dropwise and the mixture is stirred at 70° C. for 1 hour. After cooling to room temperature, the mixture is washed twice with 5% hydrochloric acid and once with water. The solvent is distilled off under reduced pressure. The crude product is fractionated under reduced pressure. 175 g (87% of theory) of 2-acetoxy-5-(trifluoromethoxy)benzyl acetate are obtained as a colourless liquid having a boiling point of 107-110° C. at 2.9 mbar.

The ¹H NMR spectrum contained the following characteristic absorptions (CDCl₃, δ/ppm): 7.32 (d, 1 H, J_{H6-H4}=2.25 Hz, H-6); 7.21 (dd, 1 H, J_{H4-H6}=2.13 Hz, J_{H4-H3}=8.88 Hz, H-4); 7.13 (d, 1 H, J_{H3-H4}=8.88 Hz, H-3); 5.06 (2, 2 H, CH₂); 2.32, 2.09 (2 s, 6 H, 2×COCH₃). The following spectrum was obtained by means of GC-MS (El, 70 eV, I/%): 292 (2, M+); 250 (54, (M-CH2CO)+); 190 (100, (M-CH2CO—HOAc)+); 162 (46, (190-CO)⁺).

### Example 7

### Preparation of 6-acetoxy-2,3,4trifluorobenzyl acetate

170 g of 6-hydroxy-2,3,4-trifluoro-N,N-dimethylbenzylamine are dissolved in 425 ml of toluene and heated to reflux. 195 ml of acetic anhydride are added dropwise and the mixture is stirred at reflux up to complete conversion. The mixture is allowed to cool to 70° C., 100 ml of methanol are added dropwise and the mixture is stirred at 70° C. for 1 hour. After cooling to room temperature, the mixture is washed twice with 5% hydrochloric acid and once with water. The solvent is distilled off under reduced pressure. The residue is added dropwise to 1000 ml of methyl tert-butyl ether, the precipitate is filtered off and the filtrate is concentrated under reduced pressure. The crude product is fractionated under reduced pressure. 109 g (50% of theory) of 6-acetoxy-2,3,4-trifluorobenzyl acetate are obtained as a colourless liquid having a boiling point of 94° C. at 1.8 mbar.

The ¹H NMR spectrum contained the following characteristic absorptions (CDCl₃, δ/ppm): 6.85 (m, 1 H, H-5); 5.12 (s, 2 H, CH2); 2.33, 2.04 (2×s, 6 H, 2×COCH₃).

The following spectrum was obtained by means of GC-MS (El, 70 eV, I/%): 262 (2, M⁺); 220 (55, (M-CH₂CO)⁺); 160 (100, (M-CH₂CO—HOAc)⁺); 132 (37, (160-CO)⁺).

### Example 8

### Preparation of 2-acetoxy-4-trifluoromethylbenzyl acetate

100 g of 2-hydroxy-4-trifluoromethyl-N,N-dimethylbenzylamine are dissolved in 180 ml of toluene and heated to reflux. 108 ml of acetic anhydride are added dropwise and the mixture is stirred at reflux up to complete conversion. The mixture is allowed to cool to 70° C., 100 ml of methanol are added dropwise and the mixture is stirred at 70° C. for 1 hour.

After cooling to room temperature, the mixture is washed twice with 5% hydrochloric acid and once with water. The solvent is distilled off under reduced pressure. The crude product is fractionated under reduced pressure. 113 g (88% of theory) of 2-acetoxy-4-trifluoromethylbenzyl acetate are obtained as a colourless liquid having a boiling point of 113-115° C. at 4.1 mbar.

The ¹H NMR spectrum contained the following characteristic absorptions (CDCl₃, δ/ppm): 7.55 (m, 2 H, H-5, H-6); 7.40 (s, 1 H, H-6); 5.10 (s, 2 H, CH2); 2.34, 2.10 (2×s, 6 H, 2×COCH₃). The following spectrum was obtained by means of GC-MS (El, 70 eV, I/%): 276 (3, M⁺); 257 (7, (M-F)⁺); 234 (84, (M-CH₂CO)⁺); 192 (32, (M-2 CH₂CO)⁺); 174 (100, (M-CH₂CO—HOAc)⁺); 146 (57, (174-CO)⁺).

### Example 9

### Preparation of 2-hydroxy-5-fluorophenylacetonitrile

151.2 g of 2-hydroxy-5-fluorobenzyl alcohol diacetate (0.66 mol) were dissolved in 500 ml of methanol and 250 ml of ethyl acetate. 81.1 g of sodium cyanide (1.65 mol, 2.5 eq) were then added. The temperature rose to 44° C. The mixture was kept at 44° C. for a further 2 h, then stirred at room temperature overnight. Subsequently, it was heated to 50° C. and stirred for a further 5.5 h.

For workup, 700 ml each of saturated NaCl solution and ethyl acetate and also 300 ml of water were added, the mixture was stirred and the phases separated. The aqueous phase was reextracted another 2× with 250 ml each time of ethyl acetate, and the combined organic phases were washed with 2×500 ml of water and with 1×500 ml of 1 N hydrochloric acid. After distilling off the solvent under reduced pressure, a black-brown oil was obtained which crystallized on cooling. 98.1 g of crude product having a purity of 77.2% by weight (HPLC-ESTD; 83 area %) (0.50 mol), corresponding to a yield of 76% of theory, were obtained.

¹H NMR (400 MHz, DMSO): δ=3.8 ppm (s, —CH₂OH); 6.85 ppm (1 Hₐᵣ), 7.0 ppm (1 Hₐᵣ), 7.1 ppm (1 Hₐᵣ); LC-MS: [M]⁺=151; 124 (—HCN); 96.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing compounds of the formula (I) 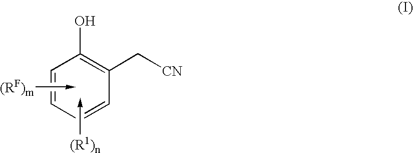
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3,
comprising
a) converting compounds of the formula (II) 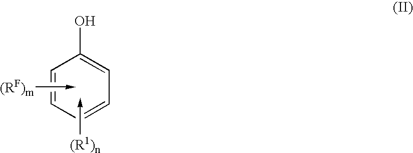
where R¹ and R^{F}, and also n and m, are as defined
in the presence of formaldehyde and
in the presence of secondary amines of the formula (III)
HNR³R⁴ (III)
where R³ and R⁴ are each independently C₁-C₈-alkyl, or NR³R⁴ as a whole is a cyclic amino radical having a total of 4 to 12 carbon atoms
to compounds of the formula (IV) 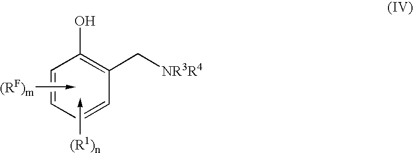
where R¹, R³, R⁴ and R^{F}, m and n, are as defined above, and
b) reacting the compounds of the formula (IV) with compounds of the formula (V)
R⁵CO—O—OCR⁵ (V)
where the R⁵ radicals are each independently hydrogen, C₁-C₁₂-alkyl, C₂-C₁₂-alkenyl, C₅-C₁₄-aryl or C₆-C₁₅-arylalkyl
to convert them to compounds of the formula (VI) 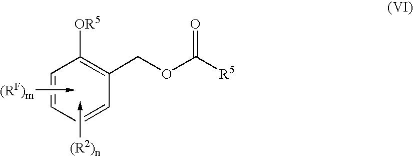
where R¹, R^{F}, m and n are each as defined under formula (I) and
the R⁵ radicals are each independently hydrogen, C₁-C₁₂-alkyl, C₂-C₁₂-alkenyl, C₅-C₁₄-aryl or C₆-C₁₅-arylalkyl, and
c) reacting the compounds of the formula (VI) with cyanide.

2. Process according to claim 1, characterized in that R¹ is in each case independently C₁-C₄-alkyl, free or protected formyl, or chlorine.

3. Process according to claim 1, characterized in that n is 0 or 1.

4. Process according to claim 1, characterized in that R^{F} is fluorine, C₁-C₄-fluoroalkyl, —O(C₁-C₄-fluoroalkyl) or —S(C₁-C₄-fluoroalkyl).

5. Process according to claim 1, characterized in that R³ and R⁴ are each an identical C₁-C₈-alkyl radical.

6. Process according to claim 1, characterized in that R⁵ is in each case identically hydrogen, C₁-C₁₂-alkyl, C₂-C₁₂-alkenyl, C₅-C₁₄-aryl or C₆-C₁₅-arylalkyl.

7. Process according to claim 1, characterized in that the molar ratio of formaldehyde to compounds of the formula (II) in step a) is 0.8 to 10.

8. Process according to claim 1, characterized in that the molar ratio of secondary amines of the formula (III) to compounds of the formula (II) in step a) is 0.8 to 10.

9. Process according to claim 1, characterized in that the molar ratio of compounds of the formula (V) to compounds of the formula (IV) in step a) is 1.5 to 10.

10. Process according to claim 1, characterized in that alkali metal cyanides are used in step c).

11. Process according to claim 1, characterized in that, in a further step d), the compounds of the formula (I) are reacted with compounds of the formulae (VIIa) or (VIIb)
R⁵CO—X (VIIa)
R⁶—Y (VIIb)
where, in formula (VIIa),
R⁵ is hydrogen, C₁-C₁₂-alkyl, C₂-C₁₂-alkenyl, C₅-C₁₄-aryl, C₆-C₁₅-arylalkyl, O(C₁-C₁₂-alkyl), O(C₅-C₁₄-aryl), O(C₆-C₁₅-arylalkyl), O(C₂-C₁₂-alkenyl), NH(C₁-C₁₂-alkyl), NH(C₅-C₁₄-aryl), NH(C₆-C₁₅-arylalkyl), N(C₁-C₁₂-alkyl)₂, N(C₅-C₁₄-aryl)₂ or N(C₆-C₁₅-arylalkyl)₂, and
X is OCOR⁵, fluorine, chlorine, bromine or iodine, and ps where, in formula (VIIb),
R⁶ is C₁-C₁₂-alkyl, C₅-C₁₄-aryl or C₆-C₁₅-arylalkyl and
Y is O₃SR⁷, chlorine, bromine or iodine where R⁷ is C₁-C₁₂-alkyl, C₅-C₁₄-aryl or C₁-C₁₂-fluoroalkyl,
to give compounds of the formula (VIII) 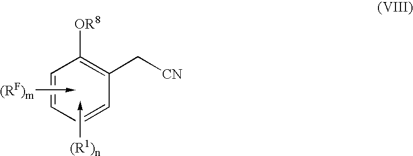
where
R⁸ is R⁵CO or R⁶ as defined above, and R¹, R^{F}, m and n are each as defined under formula (I).

12. Process of claim 1 for preparing compounds of the formula (I) 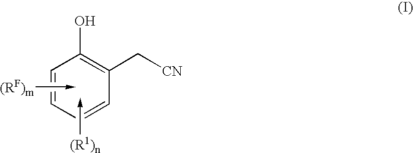
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3,
comprising reacting compounds of the formula (VI) 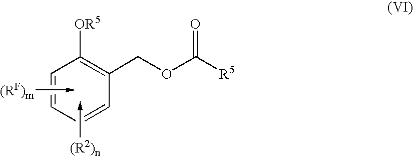
where R¹, R^{F}, m and n are each as defined under formula (I) and
the R⁵ radicals are each independently hydrogen, C₁-C₁₂-alkyl, C₂-C₁₂-alkenyl, C₅-C₁₄-aryl or C₆-C₁₅-arylalkyl with cyanide.

13. Process for preparing compounds of the formula (VI) 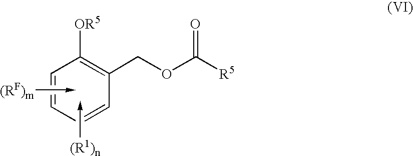
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3 and
R⁵ is in each case independently hydrogen, C₁-C₁₂-alkyl, C₂-C₁₂-alkenyl, C₅-C₁₄-aryl or C₆-C₁₅-arylalkyl,
comprising reacting compounds of the formula (IV) 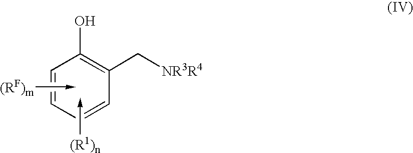
where R¹ and R^{F}, and also m and n, are as defined above and
R³ and R⁴ are each independently C₁-C₈-alkyl, or NR³R⁴ as a whole is a cyclic amino radical having a total of 4 to 12 carbon atoms with compounds of the formula (V)
R⁵CO—O—OCR⁵ (V)
where the R⁵ radicals are as defined above.

14. Process for preparing compounds of the formula (IV) 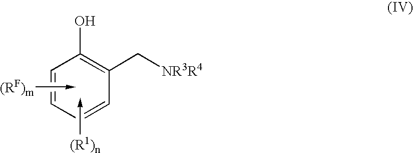
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3 and
R³ and R⁴ are each independently C₁-C₈-alkyl, or NR³R⁴ as a whole is a cyclic amino radical having a total of 4 to 12 carbon atoms,
comprising converting compounds of the formula (II) 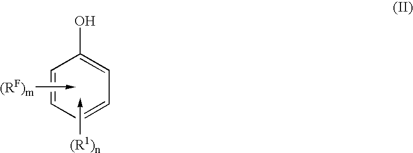
where R¹ and R^{F}, and also n and m, are as defined above
in the presence of formaldehyde and
in the presence of secondary amines of the formula (III)
HNR³R⁴ (III)
where R³ and R⁴ are as defined above.

15. Compounds of the formula (I) 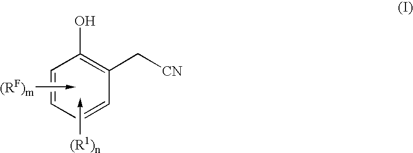
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3.

16. Compounds of formula (I) according to claim 15 selected from the group consisting of 2-hydroxy-5-fluorophenylacetonitrile, 2-hydroxy-4,5-difluorophenylacetonitrile, 2-hydroxy-5-trifluoromethoxyphenyl-acetonitrile, 6-hydroxy-2,3,4-trifluorophenylacetonitrile and 2-hydroxy-4-trifluoromethylphenylacetonitrile.

17. Compounds of the formula (IV) 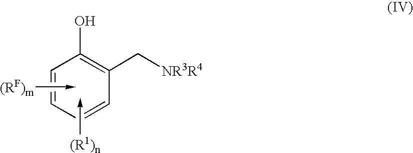
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3 and
R³ and R⁴ are each independently C₁-C₈-alkyl, or NR³R⁴ as a whole is a cyclic amino radical having a total of 4 to 12 carbon atoms,
with the exception of 2-hydroxy-5-fluoro-N,N-dimethylbenzylamine.

18. Compound of formula (IV) according to claim 17 selected from the group consisting of 4,5-difluoro-2-hydroxy-N,N-dimehtylbenzyl-amine, 2-hydroxy-5-(trifluoromethoxy)-N,N-dimethylbenzylamine, 6-hydroxy-2,3,4-trifluoro-N,N-dimethylbenzylamine and 2-hydroxy-4-(trifluoromethyl)-N,N-dimethylbenzylamine.

19. Compounds of the formula (VI) 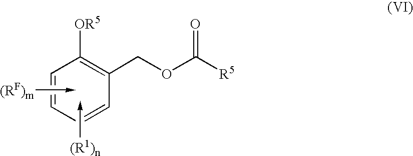
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3 and
R⁵ is in each case independently hydrogen, C₁-C₁₂-alkyl, C₂-C₁₂-alkenyl, C₅-C₁₄-aryl or C₆-C₁₅-arylalkyl, with the exception of 2-acetoxy-5-fluorobenzyl acetate.

20. Compounds of formula (VI) according to claim 17 selected from the group consisting of 2-acetoxy-4,5-difluorobenzyl acetate, 2-acetoxy-5-(trifluoromethoxy)benzyl acetate, 6-acetoxy-2,3,4-trifluorobenzyl acetate and 2-acetoxy-4-trifluoromethylbenzyl acetate.

21. Compounds of the formula (VIIIa) 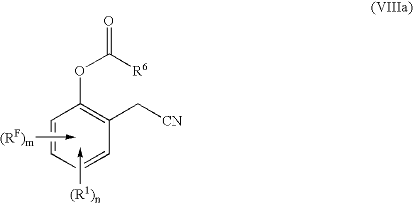
where
R¹ is in each case independently C₁-C₁₂-alkyl, free or protected formyl, chlorine or bromine or a radical of the formulae (IIa) or (IIb)
A-B-D-E (IIa)
A-E (IIb)
where, each independently,
A is absent or is a C₁-C₈-alkylene radical and
B is absent or is oxygen, sulphur or NR²
where R² is hydrogen or C₁-C₈-alkyl and
D is a carbonyl group and
E is C₁-C₈-alkyl, C₁-C₈-alkoxy, NH(C₁-C₈-alkyl) or N(C₁-C₈-alkyl)₂ or is a cyclic amino radical having 4 to 12 carbon atoms and
n is an integer of 0 to 4-m and
R^{F} is fluorine, C₁-C₁₂-fluoroalkyl, —O(C₁-C₁₂-fluoroalkyl) or —S(C₁-C₁₂-fluoroalkyl) and
m is an integer of 1 to 3 and
R⁶ is C₁-C₁₂-alkyl, C₅-C₁₄-aryl or C₆-C₁₅-arylalkyl.

22. A process for preparing active ingredients for medicaments comprising providing compounds of claim 15.

23. A process for treating cardiovascular disordersor diseases comprising administering medicaments containing active ingredients based on compounds of claim 15 to subjects in need thereof.
